(19) United States
(12) Patent Application Publication
(10) Pub. No.: US 2022/0238198 A9
(43) Pub. Date: Jul. 28, 2022

(54) PHARMACEUTICAL BUSINESS METHOD FOR HIGH VOLUME LOW RISK SALES WITH ACCELERATED MANUFACTURING

(71) Applicant: David Brent Morgan, Clinton, UT (US)

(72) Inventor: David Brent Morgan, Clinton, UT (US)

(21) Appl. No.: 17/207,636

(22) Filed: Mar. 20, 2021

Related U.S. Application Data
(63) Continuation-in-part of application No. 13/674,028, filed on Nov. 10, 2012.
(60) Provisional application No. 61/563,388, filed on Nov. 23, 2011.

(51) Int. Cl. G16H 20/10; G06Q 10/08; G06Q 10/04; G06Q 10/06; G06Q 20/40

(52) U.S. Cl. CPC G16H 20/10; G06Q 10/087; G06Q 20/401; G06Q 10/06315; G06Q 10/04

(57) ABSTRACT

A business method for pharmaceutical sales in which additional parties or entities are introduced into the transaction such that simultaneous mass manufacturing and seeking regulatory approvals for the sales of pharmaceutical is feasible. The method promotes the early widespread adoption and use of pharmaceuticals while reducing risk and increasing sales volume through guaranteed advance bulk purchase agreements. The resulting method achieves speed to mass markets heretofore not realized.

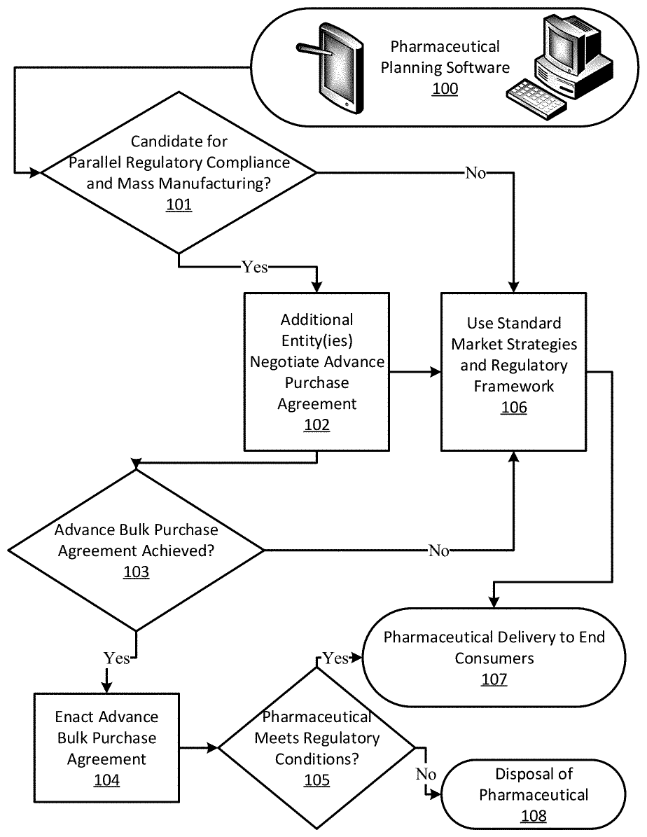

### FIELD OF THE INVENTION

The present invention pertains to the field of pharmaceutical sales software and analysis. More specifically, the invention relates to a pharmaceutical business method for high volume low risk sales with accelerated manufacturing that facilitates simultaneous mass production of a pharmaceutical while seeking regulatory approval of the pharmaceutical thus significantly decreasing the time to market using specialized software analysis tools.

### BACKGROUND

For decades the pharmaceutical industry has relied upon a serial approach to research and development and manufacturing at scale. Initial research and development and regulatory compliance costs can be staggering for the pharmaceutical industry and thus must be recaptured in a fairly short timeframe with the initial deployment of a new pharmaceutical. This serial approach has resulted in low initial sales coupled with staggering expenses thus increasing the cost of newly released pharmaceuticals for consumers and delaying time to market. While the research and development costs as well as regulatory compliance costs are eventually recaptured as the pharmaceutical matures, the pharmaceutical manufacturing side faces time to market pressure from the need to meet necessary regulatory requirements. The resulting effect is a cost and time curve such that consumers simply cannot benefit from a pharmaceutical until both regulatory and manufacturing at mass scale requirements are met. The resulting effect is that a pharmaceutical company misses a potentially large market of consumers during a crisis situation where mass quantities of the pharmaceutical are in high demand in a very short timeframe.

The dilemma of pharmaceuticals missing a potentially large market in such cases as pandemics and epidemics through invention of vaccines and therapeutic pharmaceuticals where entire countries and perhaps the global community at large are in great need of immediate vaccines as well as therapeutics to prevent and slow the spread of disease and to even prevent death until manufacturing capacity can reach a level of mass production is highly problematic. The inability of pharmaceutical companies to close the gap between early fulfilling of regulatory requirements while ramping up manufacturing capacity has become problematic to satisfy such urgent conditions.

### SUMMARY OF THE INVENTION

In accordance with the present invention, a pharmaceutical business method for high volume low risk sales with accelerated manufacturing is provided that overcomes the known problems with the gap between fulfilling regulatory requirements and having a mass supply of manufactured pharmaceuticals upon demand.

In particular, a specialized computer software analysis method for high volume low risk sales with accelerated manufacturing is provided that allows simultaneous actions of fulfilling regulatory requirements while mass manufacturing pharmaceuticals.

In accordance with an exemplary embodiment of the present invention, a pharmaceutical business method for high volume low risk sales with accelerated manufacturing pharmaceutical is provided. The method includes a specialized software analysis decision-making process aided by a computer for determining market strategy and negotiating advance purchase agreements and delivery of pharmaceuticals to end consumers.

The present invention provides many important advantages. One important advantage is the timely sales and delivery of pharmaceuticals to potential markets heretofore unrealized in the pharmaceutical market. The present invention thus allows a sustainable business model which permits near real-time access to most vital vaccines and therapeutic pharmaceuticals for large populations who would otherwise not have feasible access to such vaccines and therapeutic pharmaceuticals, resulting in the diminishing spread of disease and even untimely death.

Those skilled in the art will further appreciate the advantages and superior features of the invention together with important aspects thereof on reading the detailed description that follows in conjunction with the drawings.

### BRIEF DESCRIPTION OF THE DRAWINGS

The drawing FIGURE is a flow chart of a specialized software analysis entailing computer hardware for a pharmaceutical business method for high volume low risk sales with accelerated manufacturing in accordance with an exemplary embodiment of the present invention.

### DETAILED DESCRIPTION OF PREFERRED EMBODIMENTS

In the description which follows, like parts are marked throughout the specification and drawings with the same reference numerals respectively. The drawing FIGURE may not be to scale and certain components can be shown in generalized or schematic form and identified by commercial designation in the interest of clarity and conciseness.

The drawing FIGURE is a flow chart of a pharmaceutical business method for high volume low risk sales with accelerated manufacturing in accordance with an exemplary embodiment of the present invention. During pharmaceutical planning 100 or any time thereafter, a computer aided specialized software analysis decision making tool is utilized to determine if the pharmaceutical is a candidate for parallel regulatory compliance and mass manufacturing 101. To parallel regulatory compliance and mass manufacturing 101 one or more additional entities or parties must become involved. These entities or parties negotiate an advance purchase agreement 102 with the pharmaceutical company. A successful agreement 103 would include elements such that the additional entities or parties are able to secure a reasonable price and guarantee parallel regulatory compliance and mass manufacturing such that the pharmaceutical company could recoup necessary costs and expected profit margin if such profit is desired. Once the agreement is enacted 104 and the pharmaceutical is deemed acceptable by the appropriate regulatory body 105, delivery of the pharmaceutical could begin to the end consumers 107. If the pharmaceutical does not meet regulatory conditions; the quantities of pharmaceuticals already manufactured are appropriately disposed. If the pharmaceutical is not a good candidate for a parallel seeking regulatory compliance and mass manufacturing 101 or the advance bulk purchase agreement fails to be fully negotiated 102 or achieved 103; standard market strategies and regulatory framework 106 are pursued; standard market strategies and regulatory framework 106 could be utilized for delivery of the pharmaceutical to end consumers 107. If a pharmaceutical is deemed to have the potential of an advance purchase agreement 103 and use standard market strategies 106 the pharmaceutical has the potential to follow both paths 102.

The expected results are a pharmaceutical business method for high volume low risk sales with accelerated manufacturing. The pharmaceutical company benefits from an advance bulk volume of sales in a low risk format due to the nature of the advance bulk purchase agreement 102. Furthermore, the advance bulk purchase agreement 102 could also include provisions attractive to the pharmaceutical company to further lower risk such as a reduction in liability or guaranteed liability limit and guaranteed payment of disposed pharmaceuticals 108 in the event the pharmaceutical does not meet regulatory requirements 105.

The expected results for the end consumers, the beneficiaries of the advance bulk purchase agreement 102, are faster delivery 107 for newly developed pharmaceuticals and reduced risk. A successful and enacted advance bulk purchase agreement 104, such as in the case of a vaccine during a pandemic, will yield health benefits that would otherwise go unrealized preventing the spread of disease and even untimely death.

The term pharmaceutical company is defined herein as any party or entity that has the intent to manufacture, produce, market, or sell pharmaceuticals. The terms entity or party are defined herein as any government, business, person, association, institute, organization, non-profit, or any other organized collection of people. The term advance bulk purchase agreement is defined as any treaty, accord, arrangement, agreement, contract, bond, pledge, promise, pact, negotiation, or anything similar whether it be verbal or written for the purchase, distribution, or sales of any pharmaceutical prior to full regulatory approval of that pharmaceutical with the intent to meet regulatory requirements while mass producing the pharmaceutical in parallel. The term seeking regulatory approval is defined herein as the process by which a pharmaceutical is deemed to be safe and efficacious via clinical trials and resulting data or any other method which a regulatory body imposes on a pharmaceutical company to permit end user consumption of said pharmaceutical. The term mass manufacturing is defined herein as the ability of a pharmaceutical company to produce a pharmaceutical at sufficient scale and volume to meet required demands such as in the case of vaccine production during a global pandemic.

Although preferred and exemplary embodiments of a pharmaceutical business method for high volume low risk sales with accelerated manufacturing have been described in detail herein, those skilled in the art will also recognize that various substitutions and modification can be made to the methods without departing from the scope and spirit of the appended claims.

What is claimed is:

1. A computerized software analysis method for high volume low risk pharmaceutical sales with accelerated manufacturing; wherein mass pharmaceutical manufacturing and seeking regulatory approval occur in parallel.

2. The method as claimed in claim 1 wherein additional parties or entities negotiate an advance bulk purchase agreement.

3. The method as claimed in claim 2 wherein an advance bulk purchase agreement is successfully negotiated by said one or more additional parties or entities.

4. The method as claimed in claim 3 wherein the said bulk purchase agreement is enacted.
